SEQUENCE LISTING (2 sequences)

SEQ ID NO: 1 - protein, 174 aa, Artificial sequence
  Islet Neogenesis Associated protein (INGAP)
MLPMTLCRMSWMLLSCLMFLSWVEGEESQKKLPSSRITCPQGSVAYGSYCYSLILIPQTWSNAELSCQMHFSGHLAFLLSTGEITFVSSLVKNSLTAYQYIWIGLHDPSHGTLPNGSGWKWSSSNVLTFYNWERNPSIAADRGYCAVLSQKSGFQKWRDFNCENELPYICKFKV

SEQ ID NO: 2 - protein, 15 aa, Artificial sequence
  INGAP peptide
GLHDPSHGTLPNGSG

(19) United States
(12) Patent Application Publication
(10) Pub. No.: US 2006/0009516 A1
(43) Pub. Date: Jan. 12, 2006

(54) Use of ingap for reversing diabetes

(75) Inventor: Lawrence Rosenberg, Cote St. Luc, QC (CA)

Correspondence Address: BIRCH STEWART KOLASCH & BIRCH, PO BOX 747, FALLS CHURCH, VA, 22040-0747, US

(73) Assignee: McGill University, Montreal, QC (CA)

(21) Appl. No.: 10/532,426

(22) PCT Filed: Oct. 24, 2003
(86) PCT No.: PCT/CA03/01635

§ 371 Date: Apr. 22, 2005

Related U.S. Application Data
(60) Provisional application No. 60/420,677, filed on Oct. 24, 2002.

(51) Int. Cl. A61K 31/28

(52) U.S. Cl. 514/492

(57) ABSTRACT

The present invention relates to a method to stimulate reversal of a diabetic state in a patient; a method to prevent autoimmune destruction of new insulin-producing cells (pancreatic beta-cells) in a patient; a method to promote survival of the newly regenerated insulin-producing cells (pancreatic beta-cells); and an in vivo method for the induction of islet cell neogenesis and new islet formation and the prevention of autoimmune destruction of said new cells.

### BACKGROUND OF THE INVENTION

(a) Field of the Invention

This invention relates to a method to stimulate reversal of a diabetic state in a patient; a method to prevent autoimmune destruction of new insulin-producing cells (pancreatic beta-cells) in a patient; a method to promote survival of the newly regenerated insulin-producing cells (pancreatic beta-cells); and an in vivo method for the induction of islet cell neogenesis and new islet formation and the prevention of autoimmune destruction of said new cells; pharmaceutical compositions and uses thereof.

(b) Description of Prior Art

Diabetes

Diabetes mellitus has been classified as type I, or insulin-dependent diabetes mellitus (IDDM) and type II, or non-insulin-dependent diabetes mellitus (NIDDM). NIDDM patients have been subdivided further into (a) nonobese (possibly IDDM in evolution), (b) obese, and (c) maturity onset (in young patients). Among the population with diabetes mellitus, about 20% suffer from IDDM. Diabetes develops either when a diminished insulin output occurs or when a diminished sensitivity to insulin cannot be compensated for by an augmented capacity for insulin secretion. In patients with IDDM, a decrease in insulin secretion is the principal factor in the pathogenesis, whereas in patients with NIDDM, a decrease in insulin sensitivity is the primary factor. The mainstay of diabetes treatment, especially for type I disease, has been the administration of exogenous insulin.

Rationale for More Physiologic Therapies

Tight glucose control appears to be the key to the prevention of the secondary complications of diabetes. The results of the Diabetes Complications and Control Trial (DCCT), a multicenter randomized trial of 1441 patients with insulin dependent diabetes, indicated that the onset and progression of diabetic retinopathy, nephropathy, and neuropathy could be slowed by intensive insulin therapy (The Diabetes Control and Complication Trial Research Group, N. Engl. J. Med., 1993; 29:977-986). Strict glucose control, however, was associated with a three-fold increase in incidence of severe hypoglycemia, including episodes of seizure and coma. As well, although glycosylated hemoglobin levels decreased in the treatment group, only 5% maintained an average level below 6.05% despite the enormous amount of effort and resources allocated to the support of patients on the intensive regime (The Diabetes Control and Complication Trial Research Group, N. Engl. J. Med., 1993; 29:977-986). The results of the DCCT clearly indicated that intensive control of glucose can significantly reduce (but not completely protect against). the long-term microvascular complications of diabetes mellitus.

Other Therapeutic Options

The delivery of insulin in a physiologic manner has been an elusive goal since insulin was first purified by Banting, Best, McLeod and Collip. Even in a patient with tight glucose control, however, exogenous insulin has not been able to achieve the glucose metabolism of an endogenous insulin source that responds to moment-to-moment changes in glucose concentration and therefore protects against the development of microvascular complications over the long term.

A major goal of diabetes research, therefore, has been the development of new forms of treatment that endeavor to reproduce more closely the normal physiologic state. One such approach, a closed-loop insulin pump coupled to a glucose sensor, mimicking p-cell function in which the secretion of insulin is closely regulated, has not yet been successful. Only total endocrine replacement therapy in the form of a transplant has proven effective in the treatment of diabetes mellitus. Although transplants of insulin-producing tissue are a logical advance over subcutaneous insulin injections, it is still far from clear whether the risks of the intervention and of the associated long-term immunosuppressive treatment are lower those in diabetic patients under conventional treatment.

Despite the early evidence of the potential benefits of vascularized pancreas transplantation: it remains a complex surgical intervention, requiring the long-term administration of chronic immunosuppression with its attendant side effects. Moreover, almost 50% of successfully transplanted patients exhibit impaired tolerance curves (Wright F H et al., Arch. Surg., 1989;124:796-799; Landgraft R et al., Diabetologia 1991; 34 (suppl 1):S61; Morel P et al., Transplantation 1991; 51:990-1000), raising questions about their protection against the long-term complications of chronic hyperglycemia.

The major complications of whole pancreas transplantation, as well as the requirement for long term immunosuppression, has limited its wider application and provided impetus for the development of islet transplantation. Theoretically, the transplantation of islets alone, while enabling tight glycemic control, has several potential advantages over whole pancreas transplantation. These include the following: (i) minimal surgical morbidity, with the infusion of islets directly into the liver via the portal vein; (ii) the possibility of simple re-transplantation for graft failures; (iii) the exclusion of complications associated with the exocrine pancreas; (iv) the possibility that islets are less immunogenic, eliminating the need for immunosuppression and enabling early transplantation into non-uremic diabetics; (v) the possibility of modifying islets in vitro prior to transplantation to reduce their immunogenicity; (vi) the ability to encapsulate islets in artificial membranes to isolate them from the host immune system; and (vii) the related possibility of using xenotransplantation of islets immunoisolated as part of a biohybrid system. Moreover, they permit the banking of the endocrine cryopreserved tissue and a careful and standardized quality control program before the implantation.

The Problem of Islet Transplantation

Adequate numbers of isogenetic islets transplanted into a reliable implantation site can only reverse the metabolic abnormalities in diabetic recipients in the short term. In those that were normoglycemic post-transplant, hyperglycemia recurred within 3-12 mo. (Orloff M, et. al., Transplantation 1988; 45:307). The return of the diabetic state that occurs with time has been attributed either to the ectopic location of the islets, to a disruption of the enteroinsular axis, or to the transplantation of an inadequate islet cell mass (Bretzel R G, et al. In: Bretzel R G, (ed) Diabetes mellitus (Berlin: Springer, 1990) p.229).

Studies of the long term natural history of the islet transplant, that examine parameters other than graft function, are few in number. Only one report was found in which an attempt was specifically made to study graft morphology (Alejandro R,. et. al., J Clin Invest 1986; 78: 1339). In that study, purified islets were transplanted into the canine liver via the portal vein. During prolonged follow-up, delayed failures of graft function occurred. Unfortunately, the graft was only examined at the end of the study, and not over time as function declined. Delayed graft failures have also been confirmed by other investigators for dogs (Warnock G L et. al., Can. J. Surg., 1988; 31: 421 and primates (Sutton R, et. al., Transplant Proc., 1987; 19: 3525). Most failures are presumed to be the result of rejection despite appropriate immunosuppression.

Because of these failures, there is currently much enthusiasm for the immunoisolation of islets, which could eliminate the need for immunosuppression. The reasons are compelling. Immunosuppression is harmful to the recipient, and may impair islet function and possibly cell survival (Metrakos P, et al., J. Surg. Res., 1993; 54: 375). Unfortunately, micro-encapsulated islets injected into the peritoneal cavity of the dog fail within 6 months (Soon-Shiong P, et. al., Transplantation 1992; 54: 769), and islets placed into a vascularized biohybrid pancreas also fail, but at about one year. In each instance, however, histological evaluation of the graft has indicated a substantial loss of islet mass in these devices (Lanza R P, et. al., Diabetes 1992; 41: 1503). No reasons have been advanced for these changes. Therefore maintenance of an effective islet cell mass post-transplantation remains a significant problem.

In addition to this unresolved issue, is the ongoing problem of the lack of source tissue for transplantation. The number of human donors is insufficient to keep up with the potential number of recipients. Moreover, given the current state of the art of islet isolation, the number of islets that can be isolated from one. pancreas is far from the number required to effectively reverse hyperglycemia in a human recipient.

In response, three competing technologies have been proposed and are under development. First, islet cryopreservation and islet banking. The techniques involved, though; are expensive and cumbersome, and do not easily lend themselves to widespread adoption. In addition, islet cell mass is also lost during the freeze-thaw cycle. Therefore this is a poor long-term solution to the problem of insufficient islet cell mass. Second, is the development of islet xenotransplantation. This idea has been coupled to islet encapsulation technology to produce a biohybrid implant that does not, at least in theory, require immunosuppression. There remain many problems to solve with this approach, not least of which, is that the problem of the maintenance of islet cell mass in the post-transplant still remains. Third, is the resort to human fetal tissue, which should have a great capacity to be expanded ex vivo and then transplanted. However, in addition to the problems of limited tissue availability, immunogenicity, there are complex ethical issues surrounding the use of such a tissue source that will not soon be resolved. However, there is an alternative that offers similar possibilities for near unlimited cell mass expansion.

An entirely novel approach, proposed by Rosenberg in 1995 (Rosenberg L et al., Cell Transplantation, 1995; 4:371-384), was the development of technology to control and modulate islet cell neogenesis and new islet formation, both in vitro and in vivo. The concept assumed that (a) the induction of islet cell differentiation was in fact controllable; (b) implied the persistence of a stem cell-like cell in the adult pancreas; and (c) that the signal(s) that would drive the whole process could be identified and manipulated.

In a series of in vivo studies, Rosenberg and co-workers established that these concepts were valid in principle, in the in vivo setting (Rosenberg L et al., Diabetes, 1988; 37:334-341; Rosenberg L et al., Diabetologia, 1996; 39:256-262), and that diabetes could be reversed.

The well known teachings of in vitro islet cell expansion from a non-fetal tissue source comes from Peck and co-workers (Corneliu J G et al., Horm. Metab. Res., 1997; 29:271-277), who describe isolation of a pluripotent stem cell from the-adult mouse pancreas that can be directed toward an insulin-producing cell. These findings have not been widely accepted. First, the result has not proven to be reproducible. Second, the so-called pluripotential cells have never been adequately characterized with respect to phenotype. And third, the cells have certainly not been shown to be pluripotent.

More recently two other competing technologies have been proposed the use of engineered pancreatic p-cell lines (Efrat S, Advanced Drug Delivery Reviews, 1998; 33:45-52), and the use of pluripotent embryonal stem cells (Shambloft M J et al., Proc. Natl. Acad. Sci. USA, 1998; 95:13726-13731). The former option while attractive, is associated with significant problems. Not only must the engineered cell be able to produce insulin, but it must respond in a physiologic manner to the prevailing level of glucose- and the glucose sensing mechanism is far from being understood well enough to engineer it into a cell. Many proposed cell lines are also transformed lines, and therefore have a neoplastic potential. With respect to the latter option, having an embryonal stem cell in hand is appealing because of the theoretical possibility of being able to induce differentiation in any direction, including toward the pancreatic p-cell. However, the signals necessary to achieve this milestone remain unknown.

Islet neogenesis associated protein (INGAP) is a mediator of in vivo islet cell neogenesis from. pancreatic duct epithelial cells in several species.

It would be highly desirable to be provided with a method for the in vivo induction of re-growth of new insulin-producing cells leading to the formation of mature islets of Langerhans using INGAP peptide (the biologically active portion of the INGAP molecule), as a means of revering an established diabetic state.

Moreover, if such a diabetic was caused by pre-existing or ongoing autoimmunity, it would also be highly desirable to be provided with a method for the mitigation of such autoimmunity so that the aforementioned newly re-grown cells will not be subjected to ongoing or renewed destruction.

### SUMMARY OF THE INVENTION

One aim of the invention is to provide a method to stimulate reversal of a diabetic state in a patient.

Another aim of the invention is to provide a method to prevent autoimmune destruction of new insulin-producing cells (pancreatic beta-cells) in a patient.

Another aim of the invention is to provide a method to promote survival of the newly regenerated insulin-producing cells (pancreatic beta-cells).

Another aim of the invention is to provide an in vivo method for the induction of islet cell neogenesis and new islet formation and the prevention of autoimmune destruction of the new cells.

In accordance with the present invention there is provided a method to stimulate reversal of a diabetic state in a patient, which comprises in vivo inducing re-growth of new insulin-producing cells by administering a therapeutically effective amount of a pro-neogenesis factor to said patient, wherein formation of mature islets of Langerhans is indicative of a stimulated reversal of a diabetic state.

In accordance with the present invention there is provided a method to prevent autoimmune destruction of new insulin-producing cells (pancreatic beta-cells) in a patient, which comprises administering to said patient a therapeutically effective amount of at least one immunosuppressive agent in combination with an INGAP peptide.

In accordance with the present invention there is provided an in vivo method for the induction of islet cell neogenesis and new islet formation and the prevention of autoimmune destruction of said new cells, which comprises the steps of:
a) administering INGAP peptide to said patient in an amount sufficient to stimulate transformation of putative islet cell stem/progenitor cells in adult pancreas into islet hormone-producing cells under normal endogenous homeostatic control mechanisms, whereby cells expand in number and develop a mature glucose-sensing mechanism in a regulated manner;
b) concurrently administering to said patient at least one immunosuppressive agent in an amount sufficient to protect said islet cells from immune destruction; and
c) concurrently administering a pro-survival factor to said patient during islet cell neogenesis and new islet formation.

In accordance with the present invention there is provided an in vivo method for the induction of islet cell neogenesis and new islet formation and the prevention of autoimmune destruction of said new cells, which comprises the steps of:
a) administering INGAP peptide to said patient in an amount sufficient to stimulate transformation of putative islet cell stem/progenitor cells in adult pancreas into islet hormone-producing cells under normal endogenous homeostatic control mechanisms, whereby cells expand in number and develop a mature glucose-sensing mechanism in a regulated manner;
b) concurrently administering a pro-survival factor to said patient during islet cell neogenesis and new islet formation.

In accordance with the present invention there is provided a pharmaceutical composition for the preparation of a medicament to stimulate reversal of a diabetic state in a patient by in vivo inducing re-growth of new insulin-producing cells, which comprises a therapeutically effective amount of a pro-neogenesis factor in association with a pharmaceutically acceptable carrier.

In accordance with the present invention there is provided a pharmaceutical composition for the preparation of a medicament to prevent autoimmune destruction of new insulin-producing cells in a patient, which comprises a therapeutically effective amount of at least one immunosuppressive agent and an INGAP peptide factor in association with a pharmaceutically acceptable carrier.

In accordance with the present invention there is provided a pharmaceutical composition for the preparation of a medicament to promote survival of the newly regenerated insulin-producing cells, which comprises a therapeutically effective amount of a pro-neogenesis factor in association with a pharmaceutically acceptable carrier.

In accordance with the present invention there is provided a pharmaceutical composition for the preparation of a medicament for the induction of islet cell neogenesis and new islet formation and the prevention of autoimmune destruction of said new cells, which comprises an INGAP peptide in an amount sufficient to stimulate transformation of putative islet cell stem/progenitor cells in adult pancreas into islet hormone-producing cells under normal endogenous homeostatic control mechanisms; at least one immunosuppressive agent in an amount sufficient to protect said islet cells from immune destruction; and a pro-survival factor in association with a pharmaceutically acceptable carrier.

In accordance with the present invention there is provided a pharmaceutical composition for the preparation of a medicament for the induction of islet cell neogenesis and new islet formation and the prevention of autoimmune destruction of said new cells, which comprises an INGAP peptide in an amount sufficient to stimulate transformation of putative islet cell stem/progenitor cells in adult pancreas into islet hormone-producing cells under normal endogenous homeostatic control mechanisms; and a pro-survival factor in association with a pharmaceutically acceptable carrier.

The immunosuppressive agent includes, without limitation, sirolimus, tacrolimus, or a combination thereof.

The term “pro-neogenesis factor” is intended to mean any compounds capable of islet regeneration including, without limitation, growth factors, GLP-1, exendin-4, and an INGAP peptide.

The preferred growth factors are selected from the group consisting of insulin, IGF-I, IGF-II, EGF, Gastrin and NGF.

The term “pro-survival factor” is, intended to mean a factor including, without limitation, insulin, IGF-I, IGF-II, EGF and NGF.

The term “insulin-producing cells” is intended to mean pancreatic beta-cells.

The term “INGAP peptide” is intended to mean the fragment of native Islet Neogenesis Associated Protein (INGAP) protein which contains the biological activity of the full length molecule, including but not limited to, a biologically active fragment of:

```
(SEQ ID NO:1)
Met Leu Pro Met Thr Leu Cys Arg Met Ser Trp Met Leu Leu Ser Cys
    1 5 10 15
Leu Met Phe Leu Ser Trp Val Glu Gly Glu Glu Ser Gln Lys Lys Leu
                          20 25 30
Pro Ser Ser Arg Ile Thr Cys Pro Gln Gly Ser Val Ala Tyr Gly Ser
                  35 40 45
Tyr Cys Tyr Ser Leu Ile Leu Ile Pro Gln Thr Trp Ser Asn Ala Glu
          50 55 60
Leu Ser Cys Gln Met His Phe Ser Gly His Leu Ala Phe Leu Leu Ser
  65 70 75 80
Thr Gly Glu Ile Thr Phe Val Ser Ser Leu Val Lys Asn Ser Leu Thr
                                  85 90 95
Ala Tyr Gln Tyr Ile Trp Ile Gly Leu His Asp Pro Ser His Gly Thr
                        100 105 110
Leu Pro Asn Gly Ser Gly Trp Lys Trp Ser Ser Ser Asn Val Leu Thr
                115 120 125
Phe Tyr Asn Trp Glu Arg Asn Pro Ser Ile Ala Ala Asp Arg Gly Tyr
        130 135 140
Cys Ala Val Leu Ser Gln Lys Ser Gly Phe Gln Lys Trp Arq Asp Phe
145 150 155 160
Asn Cys Glu Asn Glu Leu Pro Tyr Ile Cys Lys Phe Lys Val,
                                165 170
```

a fragment of 15 amino acids of the sequence SEQ ID NO: 1, more precisely, such an INGAP peptide is of the following amino acid sequence: Gly Leu His Asp Pro Ser His Gly Thr Leu Pro Asn Gly Ser Gly (SEQ ID NO:2).

The term “islets of Langerhans” is intended to mean islet cells and associated cells, such as duct cells, of any origin, such as human, porcine, canine and murine, among others.

The term “neogenesis” is intended to mean the regeneration or de novo growth of cells.

Except as otherwise expressly defined herein, the abbreviations used herein for designating the amino acids and the protective groups are based on recommendations of the IUPAC-IUB Commission on Biochemical Nomenclature (Biochemistry, 1972,11:1726-1732).

### BRIEF DESCRIPTION OF THE DRAWINGS

FIG. 1 illustrates the increase in pancreatic insulin content and the reduction in the prevailing level of blood glucose resulting from the concurrent administration of INGAP peptide and sirolimus/tacrolimus and insulin in NOD mice.

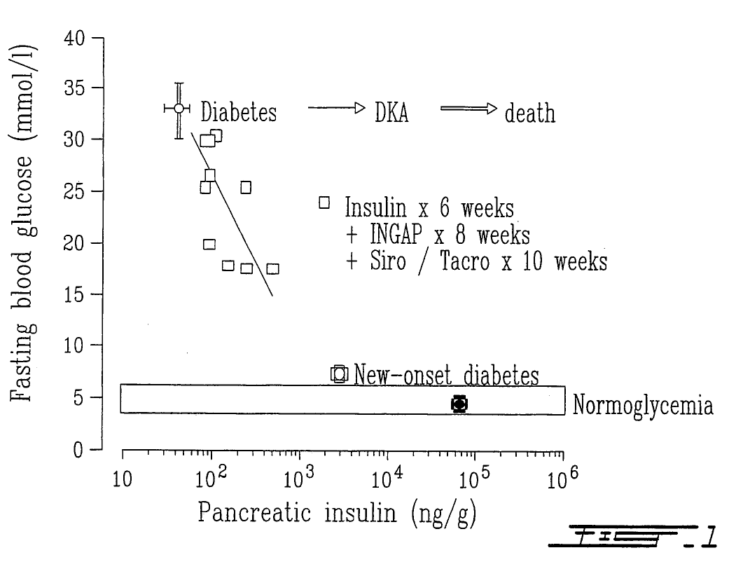

FIG. 2 illustrates the survival of NOD mice treated with a combination of INGAP peptide, sirolimus/tacrolimus and insulin versus animals treated with sirolimus/tacrolimus alone or drug vehicle alone.

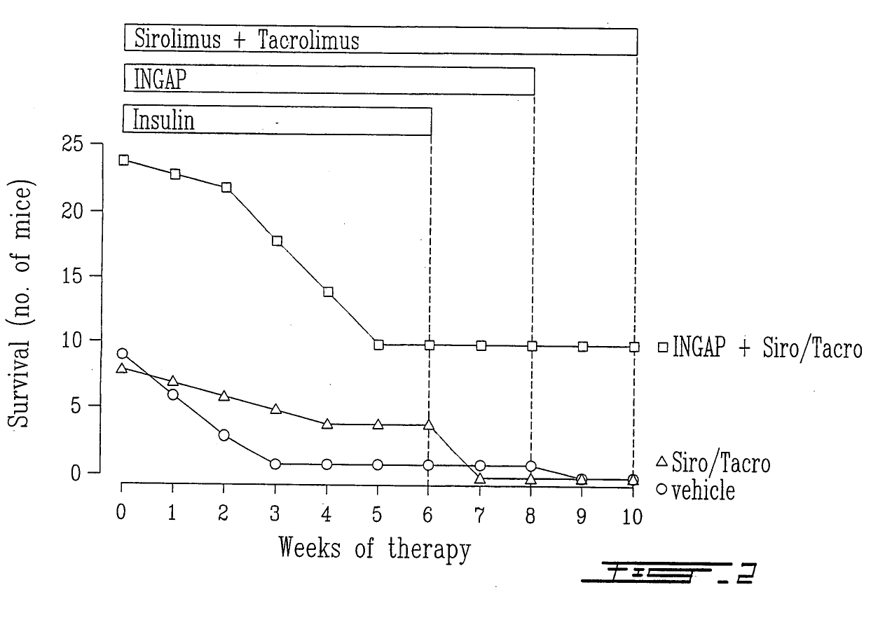

### DETAILED DESCRIPTION OF THE INVENTION

In accordance with the present invention, there is provided a method for the induction of in vivo islet cell neogenesis and new islet formation from cells derived from islet cell stem/progenitor cells in the adult pancreas, associated with the self-regulated expansion of such cells and the development of a mature glucose-sensing mechanism, leading to the reversal of an established diabetic state.

In accordance with one embodiment of the present invention, the technology is based on the understanding of autoimmune diabetes being a disease state characterized by a loss of an insulin-producing cell mass as a result of a pre-existing or ongoing autoimmune destruction of such cells, incorporating the following components that are necessary and sufficient for the successful reversal of a diabetic state by the induction of islet cell neogenesis and new islet formation:
1. a stimulus for the induction of islet cell neogenesis and new islet formation from pre-existing pancreatic stem/progenitor cells, provided by, but not limited to INGAP peptide;
2. provision of an immune tolerant environment to prevent ongoing or recurrent destruction of the newly regenerated cells, provided by, but not limited to, a combination of sirolimus/tacrolimus;
3. a pro-survival and anti-apoptosis factor, including but not limited to insulin.

The use of a pro-neogenesis factor is a critical part of the treatment, because without it, there is no stimulus to induce the transformation of putative stem/progenitor cells to new hormone-producing islet cells. Alternatively, there may be such an endogenous stimulus but it may be ineffectual in terms of overcoming a much more effective ongoing cell destruction process. Hence it is the balance of neogenesis versus destruction that may be important.

Autoimmune diabetes, by definition, occurs through the autoimmune destruction of insulin-producing pancreatic beta-cells. In order to mitigate the ongoing or renewed destruction of such cells after the induction of islet cell neogenesis, the local immune environment must be altered to remove or diminish this autoimmune insult. Thus immunosuppressive agents, that include, but are not limited to a combination of sirolimus/tacrolimus, are required.

Newly created beta-cells are known to be quite sensitive pro-death signals including, but not limited to high levels of circulating glucose. Thus pro-survival factors and in particular factors that can mitigate high levels of circulating glucose including, but not limited to insulin, are important to support and sustain cell survival.

Evidence for the induction of islet cell noegenesis and new islet formation leading to the reversal of diabetes includes: (1) an increase in the expression of the transcription factor Pdx-1 in putative islet cell progenitor cells; (2) and increase in pancreatic insulin content; (3) an increase in beta-cell mass; (5) a decrease in the prevailing level of blood glucose; (6) an increase in survival.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method to stimulate reversal of a diabetic state in a patient, which comprises in vivo inducing re-growth of new insulin-producing cells by administering a therapeutically effective amount of a pro-neogenesis factor to said patient, wherein formation of mature islets of Langerhans is indicative of a stimulated reversal of a diabetic state.

2. The method of claim 1, wherein said pro-neogenesis factor is selected from the group consisting of growth factors, GLP-1, exendin-4, and an INGAP peptide.

3. The method of claim 2, wherein said growth factor is selected from the group consisting of insulin, IGF-I, IGF-II, EGF, Gastrin and NGF.

4. The method of claim 1, wherein said insulin-producing cells are pancreatic beta-cells.

5. A method to prevent autoimmune destruction of new insulin-producing cells (pancreatic beta-cells) in a patient, which comprises administering to said patient a therapeutically effective amount of at least one immunosuppressive agent in combination with an INGAP peptide.

6. The method of claim 5, wherein said immunosuppressive agent is selected from the group consisting of sirolimus, tacrolimus, and a combination thereof.

7. A method to promote survival of the newly regenerated insulin-producing cells, which comprises administering a pro-neogenesis factor in a therapeutically effective amount to a patient.

8. The method of claim 7, wherein said pro-neogenesis factor is selected from the group consisting of growth factors, GLP-1, exendin-4, and an INGAP peptide.

9. The method of claim 8, wherein said growth factor is selected from the group consisting of insulin, IGF-I, IGF-II, EGF, Gastrin and NGF.

10. The method of claim 8, wherein said insulin-producing cells are pancreatic beta-cells.

11. An in vivo method for the induction of islet cell neogenesis and new islet formation and the prevention of autoimmune destruction of said new cells, which comprises the steps of:
a) administering INGAP peptide to said patient in an amount sufficient to stimulate transformation of putative islet cell stem/progenitor cells in adult pancreas into islet hormone-producing cells under normal endogenous homeostatic control mechanisms, whereby cells expand in number and develop a mature glucose-sensing mechanism in a regulated manner;
b) concurrently administering to said patient at least one immunosuppressive agent in an amount sufficient to protect said islet cells from immune destruction; and
c) concurrently administering a pro-survival factor to said patient during islet cell neogenesis and new islet formation.

12. The method of claim 11, wherein said islet hormone-producing cells are pancreatic beta-cells.

13. The method of claim 12, wherein said immunosuppressive agent is selected from the group consisting of sirolimus, tacrolimus, and a combination thereof.

14. The method of claim 11, wherein said pro-survival factor is selected from the group consisting of insulin, IGF-I, IGF-II, EGF and NGF.

15. An in vivo method for the induction of islet cell neogenesis and new islet formation and the prevention of autoimmune destruction of said new cells, which comprises the steps of:
a) administering INGAP peptide to said patient in an amount sufficient to stimulate transformation of putative islet cell stem/progenitor cells in adult pancreas into islet hormone-producing cells under normal endogenous homeostatic control mechanisms, whereby cells expand in number and develop a mature glucose-sensing mechanism in a regulated manner;
b) concurrently administering a pro-survival factor to said patient during islet cell neogenesis and new islet formation.

16. The method of claim 15, wherein said islet hormone-producing cells are pancreatic beta-cells.

17. The method of claim 15, wherein said pro-survival factor is selected from the group consisting of insulin, IGF-I and IGF-II.

18. A pharmaceutical composition for the preparation of a medicament to stimulate reversal of a diabetic state in a patient by in vivo inducing re-growth of new insulin-producing cells, which comprises a therapeutically effective amount of a pro-neogenesis factor in association with a pharmaceutically acceptable carrier.

19. The pharmaceutical composition of claim 18, wherein said pro-neogenesis factor is selected from the group consisting of growth factors, GLP-1, exendin-4, and an INGAP peptide.

20. The pharmaceutical composition of claim 18, wherein said growth factor is selected from the group consisting of insulin, IGF-I, IGF-II, EGF, Gastrin and NGF.

21. A pharmaceutical composition for the preparation of a medicament to prevent autoimmune destruction of new insulin-producing cells in a patient, which comprises a therapeutically effective amount of at least one immunosuppressive agent and an INGAP peptide factor in association with a pharmaceutically acceptable carrier.

22. The pharmaceutical composition of claim 21, wherein said immunosuppressive agent is selected from the group consisting of sirolimus, tacrolimus, and a combination thereof.

23. A pharmaceutical composition for the preparation of a medicament to promote survival of the newly regenerated insulin-producing cells, which comprises a therapeutically effective amount of a pro-neogenesis factor in association with a pharmaceutically acceptable carrier.

24. The pharmaceutical composition of claim 23, wherein said pro-neogenesis factor is selected from the group consisting of growth factors, GLP-1, exendin-4, and an INGAP peptide.

25. The pharmaceutical composition of claim 24, wherein said growth factor is selected from the group consisting of insulin, IGF-I, IGF-II, EGF, Gastrin and NGF.

26. The pharmaceutical composition of claim 23, wherein said insulin-producing cells are pancreatic beta-cells.

27. A pharmaceutical composition for the preparation of a medicament for the induction of islet cell neogenesis and new islet formation and the prevention of autoimmune destruction of said new cells, which comprises an INGAP peptide in an amount sufficient to stimulate transformation of putative islet cell stem/progenitor cells in adult pancreas into islet hormone-producing cells under normal endogenous homeostatic control mechanisms; at least one immunosuppressive agent in an amount sufficient to protect said islet cells from immune destruction; and a pro-survival factor in association with a pharmaceutically acceptable carrier.

28. The pharmaceutical composition of claim 27, wherein said islet hormone-producing cells are pancreatic beta-cells.

29. The pharmaceutical composition of claim 27, wherein said immunosuppressive agent is selected from the group consisting of sirolimus, tacrolimus, and a combination thereof.

30. The pharmaceutical composition of claim 29, wherein said pro-survival factor is selected from the group consisting of insulin, IGF-I, IGF-II, EGF and NGF.

31. A pharmaceutical composition for the preparation of a medicament for the induction of islet cell neogenesis and new islet formation and the prevention of autoimmune destruction of said new cells, which comprises an INGAP peptide in an amount sufficient to stimulate transformation of putative islet cell stem/progenitor cells in adult pancreas into islet hormone-producing cells under normal endogenous homeostatic control mechanisms; and a pro-survival factor in association with a pharmaceutically acceptable carrier.

32. The pharmaceutical composition of claim 31, wherein said islet hormone-producing cells are pancreatic beta-cells.

33. The pharmaceutical composition of claim 31, wherein said pro-survival factor is selected from the group consisting of insulin, IGF-I, IGF-II, EGF, and NGF.

34-49. (canceled)
